(19) United States
(12) Patent Application Publication
(10) Pub. No.: US 2026/0076658 A1
(43) Pub. Date: Mar. 19, 2026

(54) A Sprayable Patch Applicator for Stopping Haemorrhages, a Nozzle Assembly, and a Method for Applying Surgical Haemostatics to a Target Site

(71) Applicant: Ferrosan Medical Devices A/S, Søborg (DK)

(72) Inventor: Peter Lund Hammershøj, Søborg (DK)

(21) Appl. No.: 19/108,586

(22) PCT Filed: Sep. 28, 2023
(86) PCT No.: PCT/EP2023/076926

§ 371 Date: Mar. 4, 2025

(30) Foreign Application Priority Data
Sep. 30, 2022 (EP) 22199157

(51) Int. Cl. A61B 17/00; A61B 17/12

(52) U.S. Cl. CPC A61B 17/00491; A61B 2017/00522; A61B 2017/12004

(57) ABSTRACT

The invention regards a sprayable patch applicator for delivering a paste from a container, comprising: a delivery tube for connecting at a proximal end to the container, and a nozzle assembly at a distal end of the delivery tube for delivering the paste as a spray, preferably as a spray of droplets.

### FIELD OF INVENTION

The invention relates to an applicator for delivering a paste for stopping haemorrhages.

### BACKGROUND OF THE INVENTION

Surgical haemostatics are typically applied to a target site via a syringe tip. For some procedures such as minimally invasive surgical procedures, the application may occur via an extended delivery tube attached to a syringe, such that when the syringe plunger is pushed, the haemostatic is translated along the delivery tube and discharged from the opening of the delivery tube tip.

Viscous fluids, such as surgical haemostatics and haemostatic pastes, may be applied to the target site with high spatial precision. This is due to the high viscosity and/or stickiness, which facilitate that the haemostatic material is essentially extruded from the opening of the tip and applied by being deposited onto the target site. Accordingly, the tip or the opening of the tube act as an extrusion die.

For haemorrhages there is a risk of the extruded and deposited haemostatic being flushed away by the flowing blood. In such cases it may be needed to fix the haemostatic to the target site by applying additional compression force, e.g. by pressing a pad or wound dressing against the target site. This force may be applied for a while until the haemostatic is fixed, which will interrupt the surgery procedure. In addition, during laparoscopy it may difficult or impossible to provide a pad or a wound dressing at a target site.

More efficient methods and devices for applying haemostatics to a target site, and particularly target sites including haemorrhages, are therefore desired.

### SUMMARY OF THE INVENTION

Considering the prior art described above, it is an object of the present invention to provide a device that can stop haemorrhages or surface bleeds e.g. continuous oozing, problematic bleedings, difficult to access bleedings and bleedings with potential to re-bleed after surgery, preferably without having to apply manual pressure to the bleeding site.

The object can be achieved by means of an applicator according to claim 1.

Haemostatic pastes usually comprise a biocompatible polymer, such as gelatine, which provides a paste having a high viscosity with an excellent mechanical haemostatic effect. Haemostatic pastes also usually further comprise an active haemostatic agent like e.g. thrombin, which further assists in coagulating the blood.

It has been discovered that the paste can be sprayed, by a mechanism small enough to fit through a trocar, and that the paste in the form of a spray is effective on a haemorrhage.

Applying a haemostatic paste as a spray on a haemorrhage will cover a large area comprising both the wound or bleeding site and the surrounding non-bleeding area. By creating a spray of the paste, the paste will be divided in tiny droplets or microparticles providing a substantially uniform density of the paste over a larger area. The small droplets of paste formed in the spray will, when deposited on the haemorrhage and on the surrounding non-bleeding area, immediately stick together and stick to the surrounding non-bleeding area because of the high viscosity and the sticky quality of the paste. The haemostatic agent in the paste will immediately start coagulating the blood of the wound. The spray of paste will form a layer on the haemorrhage like a plaster that will stop the bleeding. A haemorrhage can be stopped by the spray of a haemostatic paste. The microparticles may comprise or contain particles smaller than 1 mm or preferably smaller than 100 μm.

The spray of paste will also have the advantage that the user will not need to apply the paste on one area and then on a neighbouring area as is done presently when applying blobs of the paste on and around the haemorrhage. The user only needs to aim the nozzle assembly towards the haemorrhage and spray the paste on the haemorrhage.

The applicator of the present disclosure is easier to apply correctly, will stop the bleeding faster, and will apply a haemostatic layer that will not be flushed away by the flowing blood.

In an embodiment, the applicator can comprise a pressure generating unit at a proximal end of the delivery tube for forcing the paste out of the container, into and through the delivery tube, to generate a spray pressure at a proximal end of the nozzle assembly.

The pressure generating unit will apply the pressure necessary for forcing the paste out of the container and into the delivery tube. The pressure generating unit can be anything that is able to pressurise a paste, like e.g. a piston pushing on the paste, a screw moving the paste forward as the screw rotates, or a high pressure fluid acting on the paste.

The delivery tube may comprise a valve for blocking paste to flow through the delivery tube from the proximal end to the distal end of the delivery tube. When the pressure on the paste in the delivery tube has been bult up by the pressure generating unit, the valve can be opened. The sudden increase in pressure through the nozzle will provide a good spray without any drooling effect—that the paste gets stuck on the outside of the applicator just outside the nozzle.

In an embodiment, the applicator can be configured such that the spray pressure is at least 2 bar, preferably at least 5 bar, more preferably at least 10 bar, even more preferably at least 20 bar, possibly even at least 25 or 30 bar. The spray pressure has to be above a smallest pressure level for producing droplets or microparticles out of the nozzle assembly. A spray pressure of at least 2 bar may produce droplets out of the nozzle assembly. With a higher spray pressure, droplets will exit the nozzle assembly even at higher flows, so that a spray pressure of at least 10 bar will provide droplets out of the nozzle even at relatively high flows, so that even a massive haemorrhage can be stopped quickly.

In an embodiment, the paste can have a viscosity of at least 100 Pa·s or between 100 Pa·s and 8000 Pa·s, preferably between 500 Pa·s and 3500 Pa·s, such as 1500 Pa·s.

In an embodiment, the applicator can be configured for delivering a spray from a paste with a viscosity of at least 500 Pa·s, or between 500 Pa·s and 8000 Pa·s, preferably between 500 Pa·s and 3500 Pa·s, such as 1500 Pa·s.

The higher the viscosity, the more sticky the paste will be. At the same time, a very viscous paste will require a higher pressure to spray out of the nozzle assembly. A viscosity above 500 Pa·s is preferable to achieve the required stickiness and 1500 Pa·s seem to be ideal balance between stickiness and spraying ability, even though the viscosity could be between 1000 and 2000 Pa·s, or 2000 and 3000 Pa·s, or 3000 and 4000 Pa·s, or 4000 and 5000 Pa·s, or 5000 and 6000 Pa·s, or higher.

For the paste to be discharged from the applicator, the paste should be flowable, when subjected to a force applicable for a syringe. Thus, by the term “flowable paste” is meant a paste having a viscosity facilitating a steady flow, when the paste is subjected to a force. An example of a flowable paste is a paste having a viscosity between 500-8000 Pa·s or between 500 and 3500 Pa·s, when measured at 30° C. and a relative humidity between 65-75%. In an embodiment of the disclosure, the paste is flowable.”The viscosity of the paste may be measured by rheometers, and preferably rotational shear based rheometers. A paste viscosity between 500-8000 Pa·s or between 500-3500 Pa·s was measured using a Discovery Hybrid Rheometer (DHR-1) from TA instruments (New Castle, DE, USA), with controlled stress and the following measurement conditions: oscillation measurement mode with time sweep, oscillation strain of 1%, angular frequency 1 rad/s, a 20 mm plate as upper geometry diameter, and a gap size of 1.25 mm. The measurements were carried out at temperatures at or between 25-30° C., and preferably at 25° C. and at a relative humidity between 65-75%.

In an embodiment, the nozzle assembly can comprise a paste inlet or nozzle assembly inlet at a proximal end and a spray outlet at a distal end of the nozzle assembly.

The paste will enter the nozzle assembly at the paste inlet or the nozzle assembly inlet as paste and leave the nozzle assembly at the spray outlet as a spray with a spray cone.

In an embodiment, the nozzle assembly can comprise a nozzle head, preferably located in extension of the distal end of the delivery tube, the nozzle head having a spray orifice at a spray outlet or the spray outlet.

The paste will leave the spray orifice or nozzle as a spray.

In an embodiment, the nozzle assembly can comprise a swirl unit for creating a rotating movement of the paste just before the paste enters the nozzle or spray orifice. The swirl unit will cause the paste to rotate around the longitudinal centre of the applicator, which will cause the paste to flow at an even higher speed, so that the spray of paste can be achieved at a lower pressure.

In an embodiment, the nozzle assembly can be configured for guiding the paste in a rotating movement around a longitudinal axis of the applicator or the nozzle assembly.

In an embodiment, the nozzle assembly can be configured to disrupt the axial flow of the paste during delivery. That the axial flow, which can mean substantially along the longitudinal axis of the applicator, of paste can be disrupted can mean that the direction is abruptly changed from an axial direction to a tangential direction. Such a disruption will cause the spray of paste to be formed at a lower pressure.

In an embodiment, the nozzle assembly can be configured to generate a full cone spray pattern when delivering the paste as a spray. Such a cone will provide an even distribution of paste over a large area of the bleeding site.

In an embodiment, the nozzle head can comprise a nozzle head tube for radially surrounding the swirl unit. The nozzle head tube when assembled or attached to the delivery tube will enable the spray orifice to be easily aligned correctly, e.g. centrally like on the longitudinal axis of the applicator.

In an embodiment, the spray orifice of the nozzle head can be elliptical, preferably circular, or polygonal, like e.g. pentagonal or hexagonal, i.e. have an elliptical, preferably circular, or polygonal, like e.g. pentagonal or hexagonal shape when seen in the distal or proximal direction.

In an embodiment, the spray orifice can be located centrally in a distal end of the nozzle head.

In an embodiment, the spray orifice can comprise a paste inlet opening inwards, wherein the paste inlet is preferably converging in the distal direction e.g. cone shaped, trumpet shaped, or parabolic, bullet-shaped in cross-section. The nozzle head surrounding the spray orifice has a certain thickness, and the paste inlet converging in the distal direction will cause the spray orifice to have a shorter length than the thickness of the nozzle head. That shorter length of the spray orifice will increase the ability of the spray orifice to provide a spray of paste out of the spray orifice.

In an embodiment, the spray orifice can comprise a spray outlet opening outwards, wherein the spray outlet is preferably diverging in the distal direction or spraying direction e.g. cone shaped, trumpet shaped, or parabolic in cross-section. The nozzle head surrounding the spray orifice has a certain thickness, and the spray outlet diverging in the distal direction will cause the spray orifice to have a shorter length than the thickness of the nozzle head. That shorter length of the spray orifice will increase the ability of the spray orifice to provide a spray of paste out of the spray orifice.

In an embodiment, wherein the spray orifice can form a double cone. The double cone can be cone shaped, trumpet shaped, or parabolic in cross-section. The nozzle head surrounding the spray orifice has a certain thickness, and the paste inlet converging in the distal direction and the spray outlet diverging in the distal direction will cause the spray orifice to have a shorter length than the thickness of the nozzle head. That shorter length of the spray orifice will increase the ability of the spray orifice to provide a spray of paste out of the spray orifice. In addition, the paste inlet and the spray outlet may form a sharp edge, where the paste inlet and the spray outlet meet. The sharp edge will provide a good spray even at low pressure and reduce the risk of drooling.

In an embodiment, the spray orifice can form a cylindrical passage, preferably distal to the paste inlet and/or preferably proximal to the spray outlet.

Alternatively, the spray orifice can be cylindrical, like fully cylindrical.

In an embodiment, the opening angle of the paste inlet and/or the spray outlet can be between 40 and 140 degrees, preferably between 60 and 120 degrees, more preferably between 80 and 100 degrees, such as around 90 degrees or around 100 degrees or around 110 degrees.

The opening angle could be understood as the angle two opposite sides or lines of the paste inlet and/or the spray outlet will form.

In one embodiment, the nozzle head may have an outside distal surface facing the distal direction and an inside proximal surface facing the proximal direction. The opening angle could be understood as the angle that the paste inlet and the distal surface of the nozzle head form. The opening angle could be understood as the angle that the proximal surface of the nozzle head and the spray outlet form.

In an embodiment, the distal end of the nozzle head can accommodate the spray orifice. The nozzle head can have a thickness of less than 2 mm, preferably less than 1.5 mm, preferably between 0.1 and 1.5 mm, more preferably between 0.6 and 1.4 mm, such as 1.0 mm. The spray orifice can have a thickness of less than 2 mm, preferably less than 1.5 mm, preferably between 0.1 and 1.5 mm, like between 0.1 and 0.5 mm or between 0.1 and 0.3 mm.

When the spray orifice is short the spray leaving the spray orifice will have a large angle so that the spray cone of paste has a large angle and a large area of the haemorrhage can be covered by the paste.

In an embodiment, the minimum diameter of the spray orifice can be less than 2 mm, preferably less than 1 mm, more preferably less than 0.6 mm, even more preferably between 0.25 mm and 0.55 mm, most preferably between 0.3 and 0.5 mm or between 0.2 and 0.5 mm or between 0.2 and 0.4 mm.

The length and diameter/width of the spray orifice can be chosen based on the paste used and based on the viscosity of the paste. A paste with a lower viscosity can preferably use a spray orifice with a longer length and/or a smaller diameter/width for avoiding leakage of paste when no or a small pressure is applied on the paste.

In an embodiment, the nozzle head can form part of the delivery tube. The nozzle head can be a continuation of the delivery tube.

In an embodiment, the nozzle head can be attached to or can be an integrated part of the distal end of the delivery tube.

In an embodiment, the cross-section of the nozzle head can be elliptical, preferably circular, polygonal, super-ellipse, or rounded rectangle.

In an embodiment, the inner diameter of the nozzle head can be less than 10 mm, preferably less than 5 mm, most preferably less than 4 mm, preferably between 2 and 4 mm, most preferably between 3 and 4 mm.

If the applicator is supposed to be used in a trocar, the inner diameter of the nozzle head could preferably be less than 4 mm, preferably between 2 and 4 mm, most preferably between 3 and 4 mm.

In an embodiment, the swirl unit can abut the nozzle head, preferably wherein the swirl unit can abut the proximal end of nozzle head. In this way, the nozzle head and the swirl unit will have a tight connection.

In an embodiment, the swirl unit can be substantially cylindrical, optionally in its entirety. In one embodiment, the swirl unit may be symmetrically positioned around a central longitudinal axis of the applicator.

In an embodiment, the swirl unit can comprise one or more axial guides, such as two, three or more axial guides, extending towards the paste inlet of the nozzle head, towards the nozzle head or in the distal direction.

In an embodiment, the axial guides can be formed as second recesses, such as longitudinal recesses, in the outer or peripheral surface of the swirl unit and/or on the inner surface of the nozzle head tube.

In an embodiment, the swirl unit and/or the proximal surface of the nozzle head can comprise one or more tangential guides or tangential channels, such as two, three or more tangential guides, towards a spray orifice in the centre of and at the distal end of the nozzle assembly.

Using two, three or more tangential guides or tangential channels means that the tangential guides or tangential channels can narrow as the tangential guides or tangential channels approach the spray orifice so that the flow velocity of paste is increased. A spray of the paste out of the spray orifice can then occur at a lower pressure applied on the paste.

In an embodiment, the tangential guides can be formed as first recesses in the distal end of the swirl unit.

In an embodiment, the tangential guides can curve inwardly towards the center of the swirl unit or the nozzle assembly.

In an embodiment, the one or more tangential guides can be extending non-radially. That the tangential guides are extending non-radially means that the tangential guides are extending a little off the position of the spray orifice inside the nozzle assembly, which will cause the paste to rotate and particularly rotate around the spray orifice before exiting the spray orifice, so that the paste achieves a higher flow or flow velocity.

In an embodiment, the second recesses forming the axial guides and/or the tangential guides can have a depth of less than 2 mm, preferably less than 1 mm, more preferably less than 0.75 mm, such as around 0.7 mm or 0.5 mm.

In an embodiment, the second recesses forming the axial guides and/or the tangential guides can have a depth of more than 0.1 mm.

In an embodiment, the axial guides and/or the tangential guides can have a depth of less than 2 mm, preferably less than 1 mm, more preferably less than 0.75 mm, such as around 0.7 mm or 0.5 mm.

In an embodiment, the axial guides and/or the tangential guides can have a depth of more than 0.1 mm.

In an embodiment, the axial guide(s) can be connected to corresponding tangential guide(s) at a distal end of the swirl unit or of the nozzle assembly.

In an embodiment, the swirl unit can comprise at a distal end a pointed tip or second protrusion, preferably cone shaped, trumpet shaped or bullet shaped. The pointed tip can preferably be in the centre of the nozzle assembly, preferably opposite the paste inlet of the spray orifice. The pointed tip will contribute to the circular movement of the paste prior to the paste entering the spray orifice, so that the flow or flow velocity (the flow and flow velocity can or cannot be understood to be interchangeable terms) is increased and a spray is formed out of the spray orifice at a lower pressure inside the applicator. The pointed tip will also direct the flow of paste toward the paste inlet of the spray orifice reducing backflow. The pointed tip provides a spray out of the spray orifice that is wide and uniform.

In an embodiment, the pointed tip can be at least partly located inside the spray orifice or configured to be at least partly located inside the spray orifice.

Such a configuration has turned out to yield a spray of paste with a wide cone.

In an embodiment, the pointed tip of the swirl unit can extend only through a part of the spray orifice.

Such a configuration has turned out to yield a spray of paste with a wide cone.

In an embodiment, the pointed tip has an opening angle or a distal angle, wherein the opening angle or the distal angle of the pointed tip can be less than 80 degrees, preferably less than 70 degrees, more preferably less than 60, most preferably around 50 degrees. In one embodiment, the opening angle or the distal angle of the pointed tip is the angle at a distal end of the pointed tip.

In an embodiment, the opening angle of the pointed tip and the paste inlet can have substantially the same angle.

In an embodiment, the opening angle of the pointed tip can be greater than the paste inlet.

In an embodiment, the opening angle of the pointed tip can be smaller than the paste inlet.

In an embodiment, the nozzle assembly can be provided at least partly or fully in metal, such as stainless steel, such as 316 stainless steel. The stainless steel nozzle assembly will be durable, can easily be sterilised and can be used many times.

In an embodiment, the nozzle assembly can be provided at least partly, preferably fully, in plastic, preferably the nozzle assembly can be injection moulded. That will be a cost-effective solution.

In an embodiment, the container can be a syringe.

In an embodiment, the delivery tube can have a length between 20-150 cm, more preferably between 25-80 cm, such as between 30-60 cm.

In an embodiment, the delivery tube can contain a volume of between 2-20 ml, or 3-18 ml, or 3-15 ml, preferably between 3-12 ml, such as 8 ml of e.g. paste.

In an embodiment, the applicator can be an endoscopic and/or laparoscopic applicator.

In an embodiment, the applicator can be adapted for insertion into a trocar, which can e.g. mean that the applicator has a length between 20-150 cm, more preferably between 25-80 cm, such as between 30-60 cm, and/or the applicator has an external and/or internal diameter of between 2-15 mm, more preferably between 3-8 mm, such as between 4-6 mm or 3-5 mm.

In an embodiment, the applicator can comprise an internal diameter of between 1-15 mm, more preferably between 2-8 mm, such as between 2-3 mm, 2-4 mm, 4-6 mm or 3-5 mm.

In an embodiment, the delivery tube can have a stiffness of above 0.5, 1.5 or 2 Gpa·m, preferably above 10 or 20 Gpa·m, and more preferably above 50 or 60 Gpa·m.

In an embodiment, the applicator may comprise a sheath enclosing the delivery tube at least partly. Preferably, the sheath encloses the proximal 90%, or the proximal 95%, or the proximal 97% of the delivery tube. Preferably, only the distal 5 cm, or the distal 4 cm of the delivery tube is not enclosed by the sheath. The sheath will enable the user to direct the applicator to the right area. Since the sheath is not enclosing the distal part of the delivery tube, the distal part of the delivery tube is manipulatable by an external robotic arm, so that the spray from the spray orifice can be directed at the haemorrhage. In one embodiment, the sheath may shorter than the delivery tube such that the distal end of the applicator can be manipulated by a robotic arm for directing a spray direction.

In an embodiment, the sheath can have a stiffness of above 0.5, 1.5 or 2 Gpa·m, preferably above 10 or 20 Gpa·m, and more preferably above 50 or 60 Gpa·m.

In an embodiment, the applicator can contain a volume of between 2-20 ml, or 3-20 ml, or 3-18 ml, or 3-15 ml, preferably between 4-12 ml, such as 8 ml or 10 ml, e.g. paste. The amount of haemostatic in the applicator should be enough to stop the haemorrhage of any cut at the surgical site. If the number of blood vessels at the surgical site are few, a smaller quantity of 1-3 ml, or 2-4 ml, like 2 ml will suffice to stop the haemorrhage of any cut at the surgical site. If the number of blood vessels at the surgical site are relatively many, a higher quantity like 5-10 ml or 8-12 ml, like 10 ml will suffice to stop the haemorrhage of any cut at the surgical site. In some cases, a higher quantity like above 10 ml may be preferable so that the applicator has enough haemostatic to be able to stop several haemorrhages without the need of refilling the applicator with new haemostatic. The surgical procedure can then be performed at a higher pace.

In an embodiment, the container or syringe comprises an volume of salt water (or another inert solution) that is substantially the same as or a little less than the volume of paste in the applicator, so that when the container is emptied, the applicator is filled with salt water and the applicator has delivered all the paste as a spray on the wound or bleeding site. No paste is wasted and no salt water will enter the wound. Since salt water is not poisonous there is no risk involved by using salt water even if salt water would enter the wound.

In an embodiment, the applicator only has a small volume for the paste and the applicator only comprises a/this small amount of paste, while the container or syringe comprises an volume of between 2-20 ml, or 3-20 ml, or 3-18 ml, or 3-15 ml, preferably between 4-12 ml, such as 8 ml or 10 ml of paste, so that that the container or syringe has provided through the applicator essentially all of the paste delivered to the wound.

In an embodiment, the applicator can comprise a material selected from the group of: metals, plastics, polymers, glass, glass fibers, carbon fibers, polymer fibers, composites such as fiber-reinforced materials, and combinations thereof.

In an embodiment, the pressure generating unit can be a piston, a spring acting on a plate positioned at a distal end of the paste, a screw for rotating at least partly inside the delivery tube or a gas pressurization unit for providing a gas with an enhanced pressure.

A spring, hydraulic fluid, pneumatic fluid can act on the piston or plate for applying a pressure on the paste. A rotating mechanism, where a rotating movement of a wheel is translated into a sliding movement of the piston. A ratcheting mechanism used in a caulking gun can also be used to move the piston.

In an embodiment, the delivery tube and the nozzle assembly can be fluidly connected by peripheral openings.

The paste will have to move tangentially to reach the nozzle or spray orifice. This will increase flow velocity of the paste and the spray of paste will be formed at a lower pressure.

In an embodiment, the nozzle assembly can comprise at a proximal end a central wall substantially perpendicular to a longitudinal axis of the applicator or the nozzle assembly, wherein the central wall has peripheral openings connecting the delivery tube and the nozzle assembly.

The central wall will guide the paste out to the periphery of the applicator so that the paste will have to move tangentially to reach the nozzle or spray orifice. A spray out of the spray orifice will be formed at lower pressure inside the nozzle assembly.

In an embodiment, the nozzle assembly can comprise a central wall dividing the delivery tube and the swirl unit, wherein the central wall has peripheral openings connecting the delivery tube and the nozzle assembly.

In an embodiment, the central wall can have a proximally directed tip or third protrusion for directing the paste towards the peripheral openings, which will lower the friction and/or will increase the flow or flow velocity of the paste through the applicator. The advantage is that the spray of paste can be formed at a lower pressure. The pressure applied on the paste in the container will be transferred to the paste positioned on the proximal side of the spray orifice with less pressure loss. The spray ca be formed at a lower applied pressure on the paste in the container, which will be more comfortable if the pressure is applied by hand or will use less energy if the pressure is applied by a machine. There will be less pressure and stress on the delivery tube.

In an embodiment, the swirl unit can be integrated in the nozzle assembly. To produce e.g. by injection moulding a single unit will be faster and therefor economically advantageous.

In an embodiment, the swirl unit can be a single unit or standalone unit. To produce e.g. by injection moulding a single unit will be faster and therefor economically advantageous.

In an embodiment, the applicator can have, fully or at least partly, an outer thickness at least in one dimension or in two dimensions of less than 15 mm, or preferably less than 7 mm, more preferably less than 6 mm, even more preferably less than 5 mm, like e.g. 5 mm or 4 mm or 3 mm.

The disclosure also relates to a nozzle assembly as described in the present application.

The disclosure also relates to a method for applying surgical haemostatics to a target site comprising the step of
providing an applicator as presented above and/or below connected to a container comprising a paste,
applying pressure on the paste in the container for feeding the paste into the applicator, and
spraying the paste through a nozzle assembly onto the target site.

In an embodiment, the pressure applied on the paste can form a spray pressure over the nozzle assembly that is at least 2 bar, preferably at least 5 bar, more preferably at least 10 bar, even more preferably at least 20 bar, possibly even at least 25 or 30 bar.

In an embodiment, the paste can have a viscosity of at least 500 Pa·s or or between 500 Pa·s and 8000 Pa·s, preferably between 500 Pa·s and 3500 Pa·s, such as 1500 Pa·s.

### DESCRIPTION OF THE DRAWINGS

The invention will in the following be described in greater detail with reference to the accompanying drawings:

FIG. 1 a schematic view of an applicator connected to a container

FIG. 2a-2c a schematic view of a first embodiment

FIG. 3a-3c a schematic view of a second embodiment

FIG. 4a-4c a schematic view of a third embodiment

FIG. 5a-5c a schematic view of a fourth embodiment

FIG. 6 a schematic view of a sixth embodiment

FIG. 7 a schematic view of a seventh embodiment

FIG. 8 a schematic view of a nozzle assembly spraying droplets of paste on a piece of paper

### DETAILED DESCRIPTION OF THE INVENTION

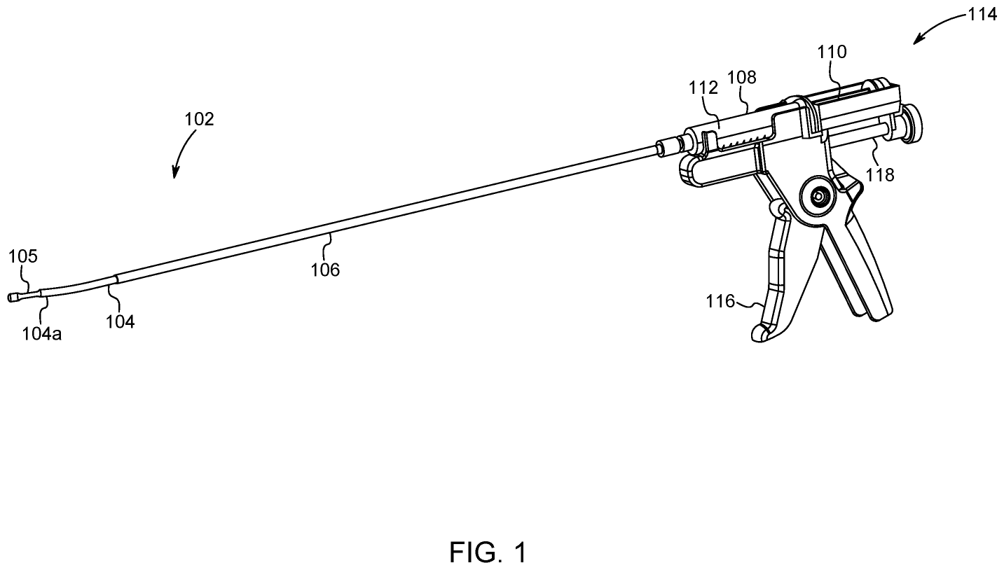

FIG. 1 shows an applicator 102 comprising a delivery tube 104 and a nozzle assembly 105, where a distal end 104a of the delivery tube 104 is connected to the nozzle assembly. The delivery tube 104 may be positioned at least partly in a rigid sheath 106 for enabling the user to direct the applicator to the right position e.g. in a patient during surgery. The applicator may preferably be not more than 5 mm in diameter to be used inside a trocar.

The applicator is connected at a proximal end of the delivery tube 104 to a container 108 e.g. a syringe. The container 108 comprises a pressure generating unit in the form of a piston 110 for applying a pressure on a paste 112 inside the container for forcing the paste out of the container and into the delivery tube and further into the nozzle assembly for generating a full cone spray pattern when delivering the paste as a spray out of the nozzle assembly.

The container 108 is positioned in an activator 114 with a trigger 116 and a rod 118, where activating the trigger will move the rod a small distance towards the piston 110. By repeatedly pressing the trigger 116, a force will be applied through the piston on the paste 112, and the paste can be sprayed out of the nozzle assembly 105.

The activator can have many designs. The activator could be electrically, e.g. by a battery (not shown) inside the activator, driven so that activating the trigger 116 activates a motor (not shown) that moves the rod 118 towards the piston 110 for spraying the paste of the applicator 102.

Instead of the piston being pushed into the container, the container could comprise a screw (not shown) that when rotated presses the paste into the delivery tube 104 for spraying the paste out of the applicator 102. The screw could be rotated by a handle (not shown) positioned off centre or by an electrically (battery) driven, rotating motor that is activated by the trigger 116.

Alternatively, a fluid activated second piston (not shown) can also be used to push on the piston 110, where a pressure source (not shown), e.g. compressor, creates a pressure that pushes the fluid activated second piston to act on the piston 110 for spraying the paste out of the applicator 102. The pressure source can be activated by activating the trigger 116, or the pressurised fluid from the pressure source can be allowed to act on the fluid activated second piston when the trigger is activated.

It is preferable that a fluid is not allowed acting on the paste directly or that a fluid is not allowed into the container 108 for avoiding the risk that the fluid accidentally leaks into the applicator and is delivered at the surgical site inside the patient.

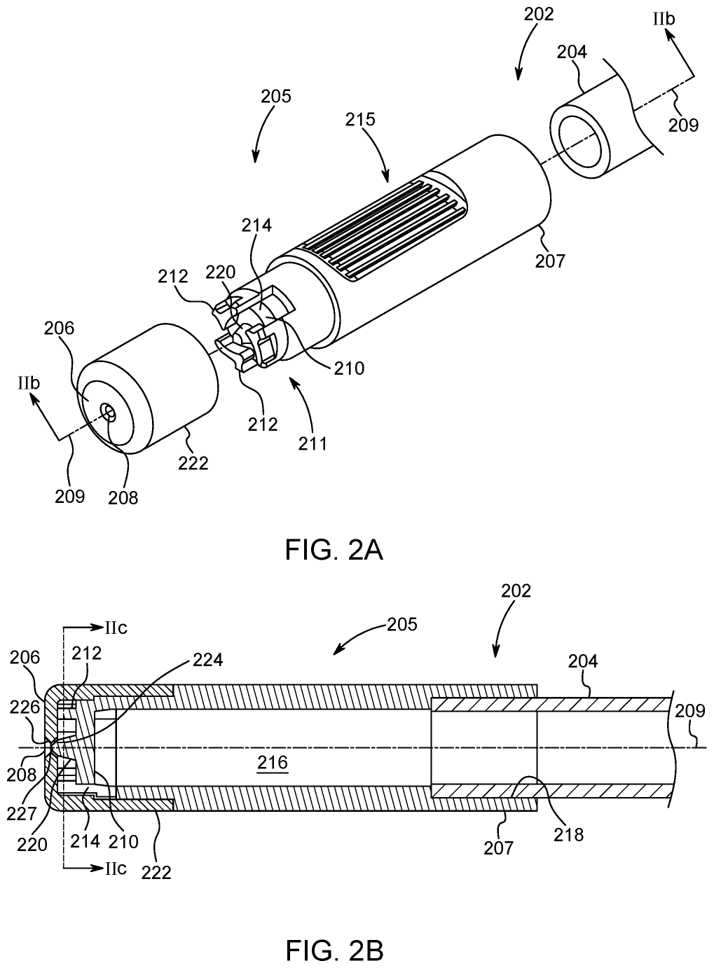

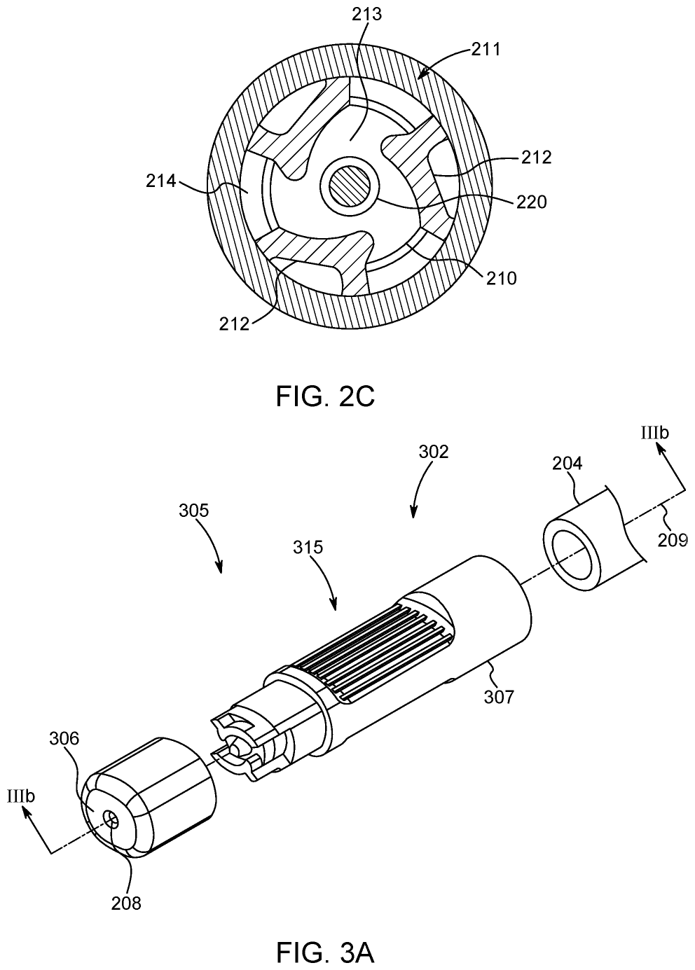

FIG. 2a-2c show a first embodiment of the applicator 202 having a delivery tube 204 (only a distal end of the delivery tube is shown) and a nozzle assembly 205. The nozzle assembly comprises a nozzle head 206 with a nozzle 208 or spray orifice 208 preferably located on a longitudinal centre axis 209 of the nozzle head, and an intermediate unit 207. The applicator 202 is preferably made of plastic.

The nozzle assembly 205 comprises a central wall 210 at a distal end of the nozzle assembly 205. A swirl unit 211 in the form of first protrusions 212 on the distal side of the central wall form tangential guides 213 or first recesses 213. The nozzle assembly 205 comprises axial guides 214 or second recesses 214 passing the central wall 210. The second recesses 214 are peripheral openings in that the second recesses are located peripheral to the central wall. The central wall 210 directs the paste out to the periphery of the applicator and through the second recesses 214. The tangential guides 213 direct the paste on the distal side of the central wall from the periphery towards the longitudinal centre axis 209 and out through the nozzle 208 as a spray.

FIG. 2a shows the first embodiment of the applicator 202 in a perspective and exploded view. The applicator 202 has two opposite third recesses 215 for allowing an easier grasp by a tweezer with jaws of an external instrument (not shown). The third recesses 215 will allow the external instrument to move the applicator to the right position at the surgical site.

A paste with a high viscosity will need a certain pressure applied on the paste before the paste will move towards the nozzle 208 and be sprayed out of the nozzle. As soon as the pressure is removed, the flow of the paste will stop. Using a paste with high viscosity a valve for stopping the flow of the paste will not be necessary. If the applicator 202 does not comprise a valve for stopping the flow of paste, and the paste will flow past the nozzle 208 even at lower pressure, the applicator 202 can be configured at the third recesses 215 to easily collapse so that flow of paste can be constricted by pressing on the third recesses 215. By clamping the third recesses 215 by the jaws of the instrument flow of paste can be reduced or even stopped.

FIG. 2b shows a cross-section along Iib in FIG. 2a of the first embodiment of the applicator 202 in an assembled view. The cross-section is in a vertical plane in FIG. 2a. The nozzle assembly 205 comprises a first cavity 216 for receiving the paste from the delivery tube 204. The nozzle assembly 205 may at a distal end have a second cavity 218 with an enlarged diameter for receiving the delivery tube 204 at a well-defined position and so that the first cavity and the delivery tube 204 have substantially the same inner diameter for allowing a movement of the paste with reduced friction. The delivery tube 204 and the nozzle assembly 205 are fixated to each, e.g. by glue or by mechanical means like e.g. by snap-fits, or the delivery tube 204 and the nozzle assembly 205 can be welded together.

FIG. 2b also shows how the nozzle head 206 and the nozzle assembly 205 are assembled. The nozzle head 206 and the nozzle assembly 205 are fastened to each other e.g. by glue or welding or mechanical means like e.g. snap-fits.

The central wall 210 may have a second protrusion 220 or a pointed tip 220 in the middle of the central wall 210 on the distal side of the central wall 210. The second protrusion 220 may be cone shaped or trumpet shaped or may have a parabolic cross-section (bullet shaped—convex cross-section). The second protrusion 220 may protrude at least partly into the nozzle 208 for improving the spray effect. The second protrusion 220 may protrude half way through the nozzle 208 for improving the spray effect. The second protrusion 220 protruding at least partly into the nozzle will lower the pressure necessary for providing the spray of paste out of the nozzle. The second protrusion 220 may protrude up to the nozzle 208, or up to 1 mm away from the nozzle, or up to 2 mm away from the nozzle.

The nozzle head 206 can be just a plate with the nozzle 208, where the nozzle head is fastened to the nozzle assembly 205 at the distal end of the nozzle assembly. However, the nozzle head 206 having a tube structure 222 will help position the nozzle 208 at the centre of the nozzle assembly 205, which will be extra beneficial when the nozzle and the second protrusion 220 are to be aligned to each other.

The nozzle 208 can have a paste inlet 224 converging in the distal direction e.g. being cone shaped, trumpet shaped, or bullet shaped. The nozzle 208 can have a spray outlet 226 diverging in the distal direction e.g. being cone shaped, trumpet shaped, or bullet shaped. The distal direction is in the spraying direction and/or in the direction of the paste during spraying. The nozzle head 206 may have an outside distal surface facing the distal direction and an inside proximal surface facing the proximal direction. Where the converging paste inlet meets the diverging spray outlet, where the converging paste inlet meets the distal surface if there is no diverging spray outlet, or where the proximal surface meets the diverging spray outlet if there is no converging paste inlet, the spray orifice will have a smallest spray orifice opening. The smallest spray orifice opening may have an orifice length of less than 0.5 mm, preferably less than 0.3 mm, like 0.2 mm or 0.1 mm. An orifice length of less than 0.5 mm will provide a spray of good quality.

In one embodiment, the second protrusion 220 may protrude at least partly into the nozzle 208 for improving the spray effect, or the second protrusion 220 may protrude at least partly into the smallest spray orifice opening for improving the spray effect even more. If the second protrusion protrudes at least partly into the nozzle or into the smallest spray orifice opening, a clearance surrounding the second protrusion 220 will be formed, where the clearance is the shortest distance between the second protrusion 220 on the one hand and the nozzle 208, the paste inlet 224, or the smallest spray orifice opening on the other hand. The clearance can preferably be less than 0.5 mm, More preferably less than 0.3 mm, even more preferably less than 0.1 mm, like 0.06 mm or 0.08 mm. With smaller clearance the spray will be broader, and the spray will comprise finer particles for faster providing a continuous and interconnected film over the wound that will stop the haemorrhage.

Alternatively or in addition to the paste inlet 224 and/or the spray outlet 226, the nozzle 208 may have a cylindrical portion 227, which is neither converging nor diverging. The cylindrical portion may have a length of at least 0.1 mm, or at least 1 mm, or at least 2 mm, or at least 3 mm, and/or the cylindrical portion may have a length of less than 10 mm, or less than 8 mm, or than 6 mm.

The first protrusions 212 may be tightened against the proximal surface of the nozzle head 206 for forming the well-defined tangential guides 213 and axial guides 214 as FIG. 2b shows.

FIG. 2c shows a cross-section along IIc in FIG. 2b. Even though FIG. 2b shows a cross-section, i.e. half of the applicator 202, the whole cross-section of the applicator is shown in FIG. 2c. FIG. 2c shows the swirl unit 211 with the first protrusions 212 forming the tangential guides 213 and the axial guides 214. In the embodiment shown in FIG. 2c, the nozzle assembly 205 has three tangential guides 213.

As the FIG. 2c shows, the axial guides 214 have a direction substantially parallel to the longitudinal axis 209 of the applicator or the nozzle assembly, and the tangential guides 213 have a direction substantially perpendicular to the longitudinal axis and/or more or less parallel to the radial direction of the nozzle assembly. However, if the tangential guides 213 are not totally parallel with the radial direction of the nozzle assembly as shown in FIG. 2c, then the moving paste will be exposed to a rotating movement in the swirl unit that will enhance the spraying ability of the paste out of the nozzle 208.

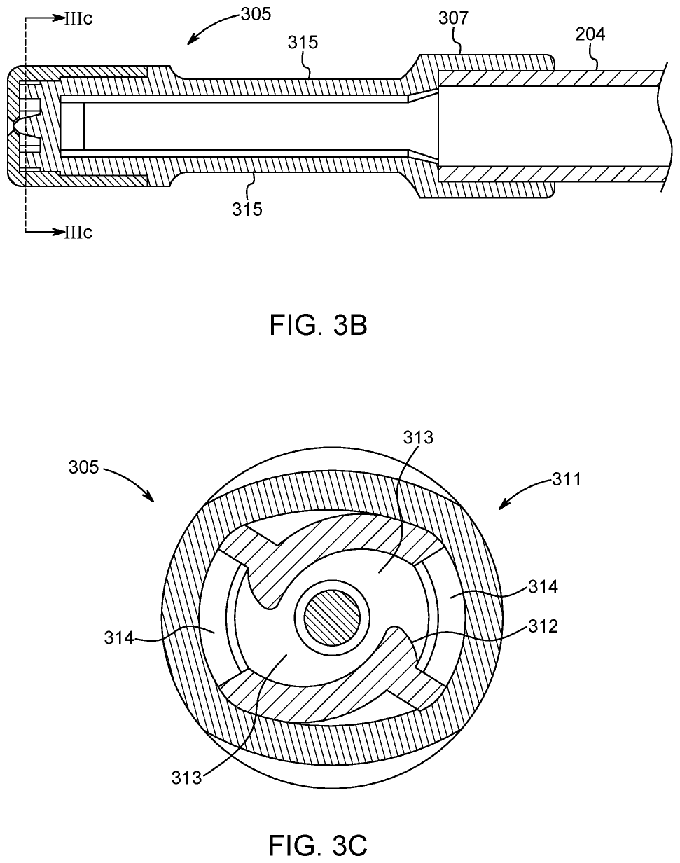

FIG. 3a-3c show a second embodiment of the applicator 302. Features of the second embodiment that have all the qualities and functionalities as the corresponding features in the first embodiment are given the same numbers.

The applicator 302 has a delivery tube 204 and a nozzle assembly 305. The nozzle assembly comprises a nozzle head 306 with a nozzle 208 preferably on a longitudinal centre axis 209 of the nozzle head, and an intermediate unit 307. The applicator 302 is preferably made of plastic.

The second embodiment of the applicator 302 can have any, any combination, or all the features and advantages as mentioned above regarding the first embodiment. The differences between the second embodiment and the first embodiment are that the nozzle assembly 305 has a somewhat flattened cross-section with a square or substantially rectangular shape resulting in smaller thickness at least in one dimension that means that the third recesses 315 can be even easier to grasp by an external instrument with a jaws, since the thickness over the third recesses 315 is smaller in the second embodiment than of the first embodiment.

FIG. 3a shows the second embodiment of the applicator 302 in a perspective and exploded view. Because of the somewhat flattened cross-section of the applicator, the thickness of the third recesses 315 can be made even slimmer so that jaws of a grasping instrument can easier clamp the applicator 302 at the third recesses 315 of the second embodiment than the of the first embodiment.

FIG. 3b shows a cross-section along IIIb in FIG. 3a of the second embodiment of the applicator 302 in an assembled view. The cross-section is in a vertical plane in FIG. 3a.

FIG. 3c shows a cross-section along IIIc in FIG. 3b. Even though FIG. 3b shows a cross-section, i.e. half of the applicator 302, the whole cross-section of the applicator is shown in FIG. 3c. FIG. 3c shows the swirl unit 311 with the first protrusions 312 forming the tangential guides 313 and the axial guides 314. Apart from the first embodiment, the nozzle assembly 305 of the second embodiment shown in FIG. 3c, has two tangential guides 313.

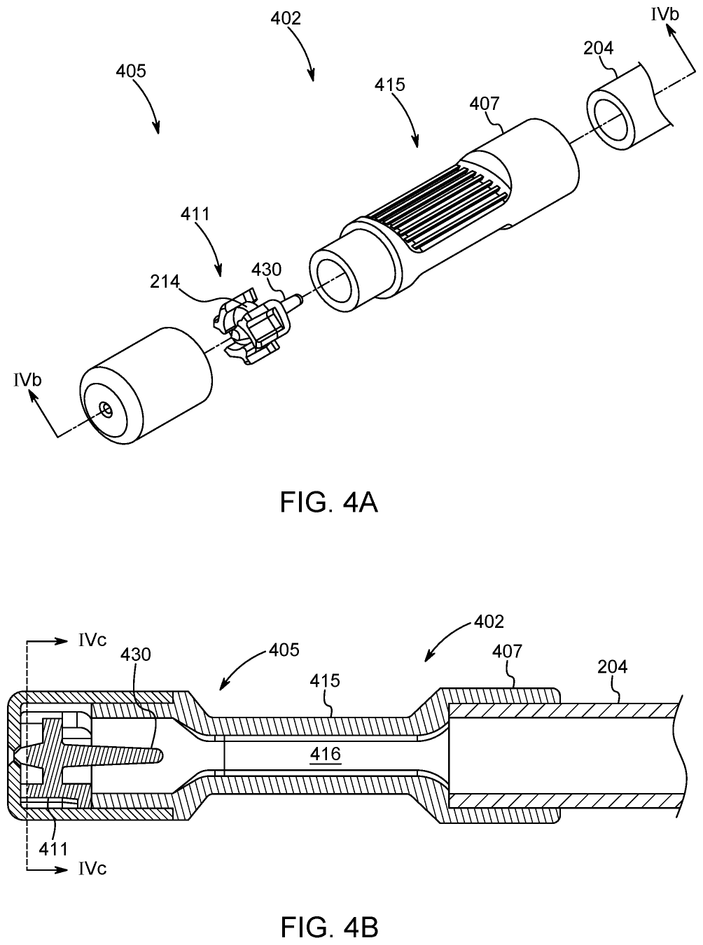

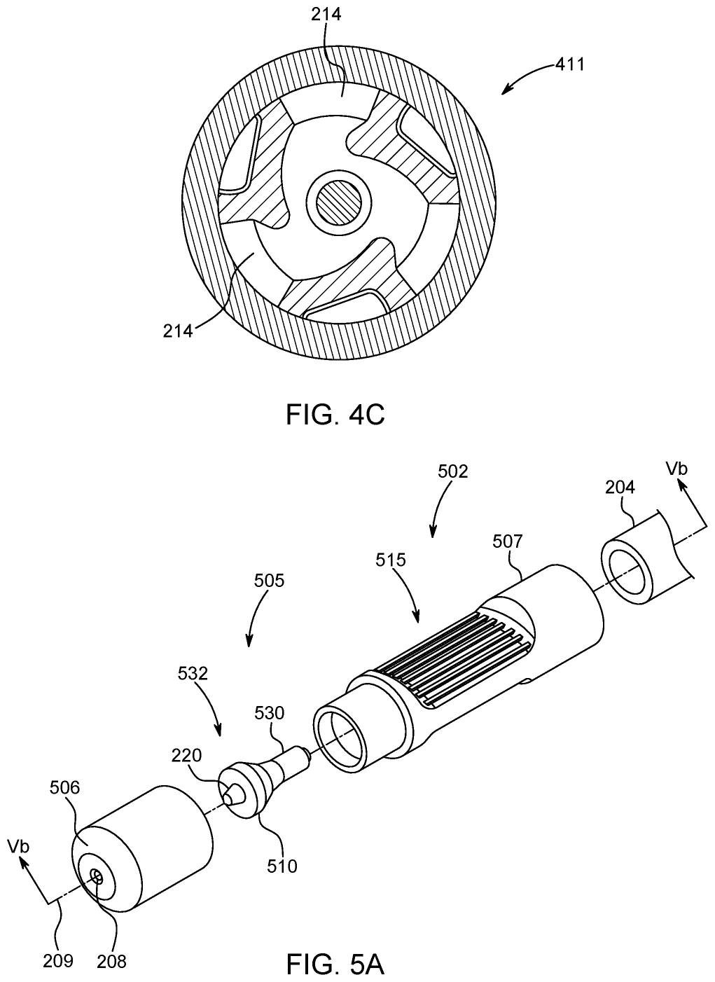

FIG. 4a-4c show a third embodiment of the applicator 402. Features of the third embodiment that have all the qualities and functionalities as the corresponding features in the first embodiment are given the same numbers.

The applicator 402 has a delivery tube 204 and a nozzle assembly 405. The nozzle assembly comprises a nozzle head 406 with a nozzle 208 preferably on a longitudinal centre axis 209 of the nozzle head, and an intermediate unit 407. The applicator 402 is preferably made of plastic.

The third embodiment of the applicator 402 can have any, any combination, or all the features and advantages as mentioned above regarding the first or the second embodiment. The differences between the third embodiment and the first embodiment are that the swirl unit 411 is a single unit or standalone unit that is not part of the rest of the nozzle assembly 405, that the swirl unit has a third protrusion 430 on the proximal side of the swirl unit, and that the first cavity 416 has a reduced inner diameter at least along a part of the first cavity so that the thickness of the nozzle assembly 405 at the two opposite third recesses 415 can be reduced even further and jaws of a clamping instrument can easier clamp the two opposite third recesses 415.

FIG. 4a shows the third embodiment of the applicator 402 in a perspective and exploded view. The third protrusion 430 will make it easier for a human or a robot assembling the applicator 402 to hold to the swirl unit 411. The third protrusion 430 may also increase the flow of paste at the transition from the first cavity 416 to the axial guides 214 by guiding the paste towards the axial guides 214 and reducing backpressure. The third protrusion may be positioned on a proximal side of the central wall with or without the swirl unit.

FIG. 4b shows a cross-section along Ivb in FIG. 4a of the third embodiment of the applicator 402 in an assembled view, where reduced inner diameter of the first cavity 416 is clearly seen. The cross-section is in a vertical plane in FIG. 4a.

FIG. 4c shows a cross-section along Ivc in FIG. 4b. Even though FIG. 4b shows a cross-section, i.e. half of the applicator 402, the whole cross-section of the applicator is shown in FIG. 4c. FIG. 4c shows the swirl unit 411, which has the same design as the swirl unit 211 in FIG. 2c when seen from this direction.

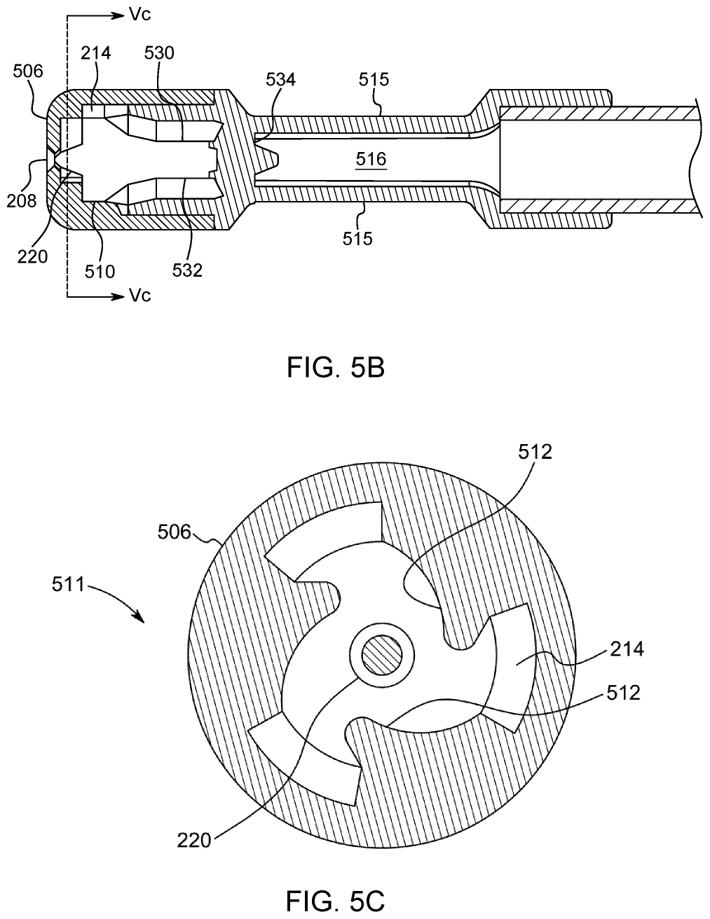

FIG. 5a-5c show a fourth embodiment of the applicator 502. Features of the fourth embodiment that have all the qualities and functionalities as the corresponding features in the first embodiment are given the same numbers.

The applicator 502 has a delivery tube 204 and a nozzle assembly 505. The nozzle assembly comprises a nozzle head 506 with a nozzle 208 preferably on a longitudinal centre axis 209 of the nozzle head, and an intermediate unit 507. The applicator 502 is preferably made of plastic.

The fourth embodiment of the applicator 502 can have any, any combination, or all the features and advantages as mentioned above regarding any of the first, second and third embodiments. The differences between the fourth embodiment and the first embodiment are that the first protrusions 512 is an integrated part of or is moulded together with the nozzle head 506, and the central wall 510, the second protrusion 220 or the pointed tip, and a third protrusion 530 on the proximal side of the central wall form a first single unit 532 or standalone unit, that the first cavity 516 has a reduced inner diameter at least along a part of the first cavity so that the thickness of the nozzle assembly 505 at the two opposite third recesses 515 can be reduced even further and jaws of a clamping instrument can easier clamp the two opposite third recesses 515.

FIG. 5a shows the fourth embodiment of the applicator 502 in a perspective and exploded view. The third protrusion 530 will make it easier for human or a robot assembling the applicator 502 to hold to the first single unit 532. The third protrusion 530 may also increase the flow of paste at the transition from the first cavity 516 to the axial guides 214 and passed the central wall 510 by guiding the paste towards the periphery of the central wall and to the axial guides 214 and reducing backpressure. The third protrusion may be positioned on a proximal side of the central wall with or without the swirl unit. The intermediate unit 507 comprises a second central wall 534 aligning the first single unit 532 through the third protrusion 530. There are channels (not shown) passed the second central wall 534 for guiding the paste passed the second central wall 534.

FIG. 5b shows a cross-section along Vb in FIG. 5a of the fourth embodiment of the applicator 502 in an assembled view, where reduced inner diameter of the first cavity 516 is clearly seen. The cross-section is in a vertical plane in FIG. 5a.

FIG. 5c shows a cross-section along Vc in FIG. 5b. Even though FIG. 5b shows a cross-section, i.e. half of the applicator 502, the whole cross-section of the applicator is shown in FIG. 5c. FIG. 5c shows the swirl unit 511, which is part of the nozzle head 506, and the second protrusion 220, which is part of the first single unit 532.

The fourth embodiment can otherwise have any, any combination, or all the features of any of the first, second and third embodiments.

In a fifth embodiment (not shown) of the applicator, the nozzle head is an integrated part of or is moulded together with the intermediate unit. The swirl unit is a single unit that will be introduced into the first cavity from the proximal end of the intermediate unit. The swirl unit and possibly the second protrusion will be well aligned with the nozzle and the spray orifice.

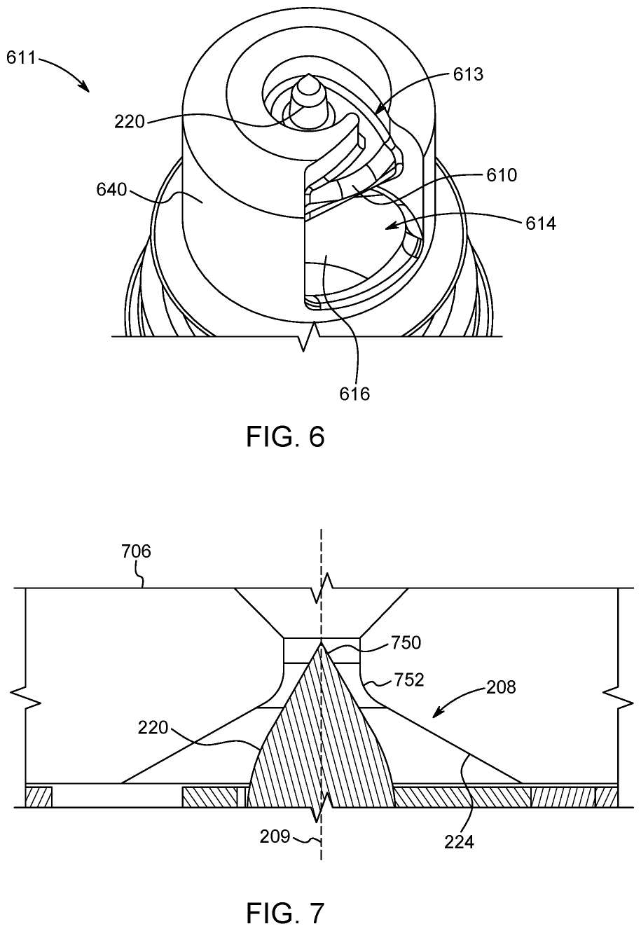

FIG. 6 shows a distal end of an example of a sixth embodiment of the applicator. In the sixth embodiment of the applicator, the swirl unit 611 may comprise a single second recess 614 and a single tangential guide 613, as shown in FIG. 6, so that the paste can be guided from the first cavity 616, passed the central wall 610, and into and around the second protrusion 220. The nozzle head (not shown), which in the shown embodiment is supposed to have the tube structure (not shown) for aligning the nozzle head around the swirl unit by sliding the nozzle head down over an outer surface 640, so that the nozzle (not shown) can be correctly oriented in relation to and aligned with the second protrusion 220. The sixth embodiment without the tube structure could also be contemplated.

With the single second recess 614 and the single tangential guide 613, any blockage between and including the first cavity 616 and the nozzle (not shown) can be removed by applying pressure on the paste by the pressure generating unit (not shown). There is no alternative second recess and/or single tangential guide, which the paste can pass through, so that the blockage is not cleared.

In FIG. 6, the second protrusion base width 642 of the second protrusion is shown. The second protrusion base width 642 is the width of the second protrusion, where the second protrusion is attached to the central wall 610. In one embodiment, the second protrusion base width can be less than 1 mm, preferably less than 0.8 mm, more preferably less than 0.6 mm, like between 0.2 and 0.5 mm.

The sixth embodiment can have any, any combination of, or all the features of any of the first, second, third, fourth, and fifth embodiments.

In a seventh embodiment, the second protrusion 220 may protrude into the nozzle 208 of the nozzle head 706, as shown in FIG. 7.

In the seventh embodiment, the second protrusion 220 may have a second protrusion point 750, preferably pointing in the distal direction, with a second protrusion point angle of between 30° and 90°, preferably between 40° and 80°, more preferably between 50° and 70°, like 60°. The second protrusion 220 protruding into the nozzle 208 and the paste inlet 224 may have opposite surfaces that may be at least partly parallel for providing a clearance between the opposite surfaces with a constant width. The two opposite surfaces may form a clearance in the form of a truncated cone with a constant thickness depending on the distal-proximal position. Due to the shape of the truncated cone, the volume for the paste moving from the proximal end to the distal end of the opposite surfaces will decrease so that the flow velocity of the paste will increase when approaching the second protrusion point 750, which will increase the spray quality, i.e. a spray with smaller particles and a wider spray cone.

The two opposite surfaces may form a clearance in the form of a truncated cone with varying thickness depending on the distal-proximal position. In the example shown in FIG. 7, the opposite surfaces form in the proximal end a clearance that decreases in the distal direction until a waist clearance 752 is reached, where the clearance has its narrowest passage. On the distal side of the waist clearance, the width of the clearance increases. The decreasing clearance until the waist clearance 752 will increase the flow velocity of the paste considerably for providing a spray of good quality.

The waist clearance 752 may be positioned at the very distal end of the second protrusion point 750, so that on the distal side of the waist clearance there is no second protrusion point 750. The paste leaving the clearance will leave the clearance at the highest flow velocity that will provide a spray of good quality. The longitudinal axis 209 is also shown for showing the centre axis of the nozzle 208 and the second protrusion point 750.

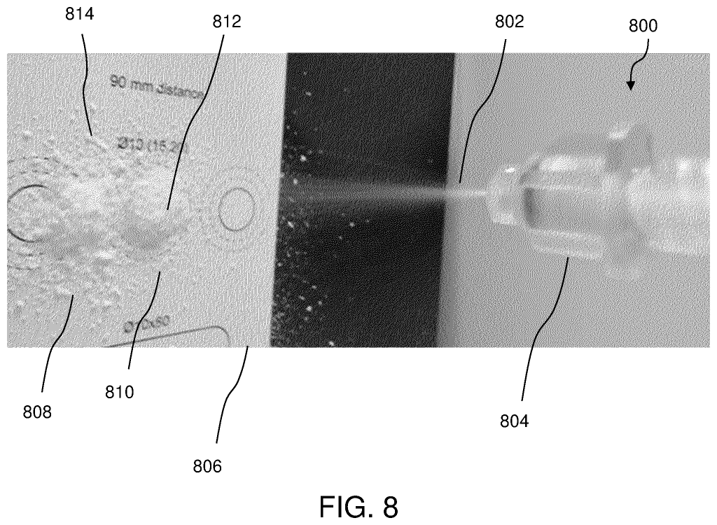

An applicator 800 in action is shown in FIG. 8. A spray cone 802 is exiting a nozzle assembly 804 with a spray angle of somewhere between 20° to 40°. The spray is in the present example deposited on a plate 806 around 90 mm away from the nozzle assembly.

A first deposit 808 has been formed. Subsequently, the nozzle assembly has been moved and/or rotated for forming a second deposit 810. As appears from the example, the second deposit has a highest deposition rate at a centre 812 of the second deposit but still covers a relatively large peripheral area 814 around the centre. So the applicator 800 in the present example will be able to both stop a severe haemorrhage and at the same time stop a haemorrhage covering a larger area.

### Items

1. An applicator for delivering a paste from a container, comprising:
a delivery tube for connecting or configured to connect at a proximal end to the container, and
a nozzle assembly at a distal end of the delivery tube for delivering the paste as a spray, preferably as a spray of droplets or of microparticles.
2. The applicator according to item 1, wherein the applicator comprises the container, and wherein the applicator comprises a pressure generating unit at a proximal end of the delivery tube for forcing the paste out of the container, into and through the delivery tube, to generate a spray pressure at a proximal end of the nozzle assembly.
3. The applicator according to item 2, configured such that the spray pressure is at least 2 bar, preferably at least 5 bar, more preferably at least 10 bar, even more preferably at least 20 bar, possibly even at least 25 or 30 bar.
4. The applicator according to any of the preceding items, wherein the paste has a viscosity of at least 500 Pa·s or between 500 Pa·s and 8000 Pa·s, preferably between 500 Pa·s and 3500 Pa·s, such as 1500 Pa·s and/or wherein the applicator is configured for delivering a spray from a paste with a viscosity of at least 500 Pa·s, or a viscosity between 500 Pa·s and 8000 Pa·s, preferably between 500 Pa·s and 3500 Pa·s, such as 1500 Pa·s.
5. The applicator according to any of the preceding items, wherein the nozzle assembly comprises a paste inlet or a nozzle assembly inlet at a proximal end and a spray outlet at a distal end of the nozzle assembly.
6. The applicator according to any of the preceding items, wherein the nozzle assembly comprises a nozzle head, preferably located in extension of the distal end of the delivery tube, the nozzle head having a spray orifice at a spray outlet.
7. The applicator according to any of the preceding items, wherein the nozzle assembly comprises a swirl unit optionally for creating a rotating movement of the paste.
8. The applicator according to any of the preceding items, wherein the nozzle assembly is configured for guiding the paste in a rotating movement around a longitudinal axis of the applicator or the nozzle assembly.
9. The applicator according to any of the preceding items, wherein the nozzle assembly is configured to disrupt the axial flow of the paste during delivery.
10. The applicator according to any of the preceding items, wherein the nozzle assembly is configured to generate a full cone spray pattern when delivering the paste as a spray.
11. The applicator according to any of the preceding items, wherein the nozzle head comprises a nozzle head tube for radially surrounding the swirl unit.
12. The applicator according to any of the preceding items 6-11, wherein the spray orifice of the nozzle head is elliptical, preferably circular, or polygonal, like e.g. pentagonal or hexagonal.
13. The applicator according to any of the preceding items 6-12, wherein the spray orifice is located centrally in a distal end of the nozzle head.
14. The applicator according to any of the preceding items 6-13, wherein the spray orifice comprises a paste inlet opening inwards, wherein the paste inlet is preferably converging in the distal direction e.g. cone shaped, trumpet shaped, or parabolic in cross-section.
15. The applicator according to any of the preceding items 6-14, wherein the spray orifice comprises a spray outlet opening outwards, wherein the spray outlet is preferably diverging in the distal direction or spraying direction e.g. cone shaped, trumpet shaped, or parabolic in cross-section.
16. The applicator according to any of the preceding items 6-15, wherein the spray orifice forms a double cone, double trumpet, or double parabolic, preferably with a waist.
17. The applicator according to any of the preceding items 6-16, wherein the spray orifice forms a cylindrical passage, preferably distal to the paste inlet and/or preferably proximal to the spray outlet.
18. The applicator according to any of the preceding items 6-17, wherein the opening angle of the paste inlet and/or the spray outlet is between 40 and 140 degrees, preferably between 60 and 120 degrees, more preferably between 80 and 100 degrees, such as around 90 degrees.
19. The applicator according to any of the preceding items 6-18, wherein the distal end of the nozzle head accommodating the spray orifice has a thickness of less than 2 mm, preferably less than 1.5 mm, preferably between 0.1 and 1.5 mm, more preferably between 0.6 and 1.4 mm, such as 1.0 mm.
20. The applicator according to any of the preceding items 6-19, wherein the spray orifice has a length of less than 2 mm, preferably less than 1.5 mm, preferably between 0.1 and 1.5 mm, more preferably between 0.6 and 1.4 mm, such as 1.0 mm.
21. The applicator according to any of the preceding items 6-20, wherein the minimum diameter of the spray orifice is less than 2 mm, preferably less than 1 mm, more preferably less than 0.6 mm, even more preferably between 0.25 mm and 0.55 mm, most preferably between 0.3 and 0.5 mm.
22. The applicator according to any of the preceding items 6-21, wherein the nozzle head forms part of the delivery tube.
23. The applicator according to any of the preceding items 6-22, wherein the nozzle head is attached to or is an integrated part of the distal end of the delivery tube.
24. The applicator according to any of the preceding items 6-23, wherein the cross-section of the nozzle head is elliptical, preferably circular, polygonal, super-ellipse, or rounded rectangle.
25. The applicator according to any of the preceding items 6-24, wherein the inner diameter of the nozzle head is less than 10 mm, preferably less than 5 mm, most preferably less than 4 mm, preferably between 2 and 4 mm, most preferably between 3 and 4 mm.
26. The applicator according to any of the preceding items 7-25, wherein the swirl unit abuts the nozzle head, preferably wherein the swirl unit abuts the proximal end of nozzle head.
27. The applicator according to any of the preceding items 7-26, wherein the swirl unit is substantially cylindrical.
28. The applicator according to any of the preceding items 7-27, wherein the swirl unit comprises one or more axial guides, such as two, three or more axial guides, extending towards a paste inlet.
29. The applicator according to any of the preceding items 7-28, wherein the axial guides are formed as second recesses, such as longitudinal recesses, in the outer surface of the swirl unit or on the distal surface of the nozzle head.
30. The applicator according to any of the preceding items 7-29, wherein the swirl unit and/or the distal surface of the nozzle head comprise(s) one or more tangential guides or tangential channels, such as two, three or more tangential guides, for guiding paste towards a spray outlet.
31. The applicator according to any of the preceding items 7-30, wherein the tangential guides are formed as first recesses in the distal end of the swirl unit.
32. The applicator according to any of the preceding items 7-31, wherein the tangential guides are curved, optionally inwardly, optionally towards the center of the swirl unit.
33. The applicator according to any of the preceding items, wherein the one or more tangential guides are extending non-radially.
34. The applicator according to any of the preceding items, wherein the second recesses forming the axial guides and/or the first recesses forming the tangential guides have a depth of less than 2 mm, preferably less than 1 mm, more preferably less than 0.75 mm, such as around 0.7 mm or 0.5 mm.
35. The applicator according to any of the preceding items, wherein the axial guides and/or the tangential guides has/have a depth of less than 2 mm, preferably less than 1 mm, more preferably less than 0.75 mm, such as around 0.7 mm or 0.5 mm.
36. The applicator according to any of the preceding items 7-35, wherein the axial guide(s) is/are connected to corresponding tangential guide(s) at a distal end of the swirl unit and/or at a distal end of the axial guide(s).
37. The applicator according to any of the preceding items 7-36, wherein the swirl unit comprises at a distal end a pointed tip or second protrusion, preferably cone shaped, trumpet shaped or bullet shaped.
38. The applicator according to item 37, wherein the pointed tip is at least partly located inside the spray orifice or configured to be at least partly located inside the spray orifice.
39. The applicator according to any of the preceding items 37-38, wherein the pointed tip of the swirl unit only extends through a part of the spray orifice.
40. The applicator according to any of the preceding items 37-39, wherein the opening angle of the pointed tip is less than 80 degrees, preferably less than 70 degrees, more preferably less than 60, most preferably around 50 degrees.
41. The applicator according to any of the preceding items 37-40, wherein the opening angle of the pointed tip and the paste inlet have substantially the same angle.
42. The applicator according to any of the preceding items 37-41, wherein the opening angle of the pointed tip is greater than the paste inlet.
43. The applicator according to any of the preceding items 37-42, wherein the opening angle of the pointed tip is smaller than the paste inlet.
44. The applicator according to any of the preceding items, wherein the nozzle assembly is provided at least partly in metal, such as stainless steel, such as 316 stainless steel.
45. The applicator according to any of the preceding items, wherein the nozzle assembly is provided at least partly, preferably fully, in plastic, preferably the nozzle assembly is injection molded.
46. The applicator according to any of the preceding items, wherein the container is a syringe.
47. The applicator according to any of the preceding items, wherein the delivery tube has a length between 20-150 cm, more preferably between 25-80 cm, such as between 30-60 cm.
48. The applicator according to any of the preceding items, wherein the delivery tube contains a volume of between 2-20 ml, preferably between 3-12 ml, such as 8 ml.
49. The applicator according to any of the preceding items, wherein the applicator is an endoscopic and/or laparoscopic applicator.
50. The applicator according to any of the preceding items, wherein the applicator is adapted for insertion into a trocar.
51. The applicator according to any of the preceding items, wherein the applicator comprises an internal diameter of between 1-15 mm, more preferably between 2-8 mm, such as between 2-3 mm, 2-4 mm, 4-6 mm or 3-5 mm.
52. The applicator according to any of the preceding items, wherein the delivery tube has a stiffness of above 0.5, 1.5 or 2 GPa·m, more preferably above 50 or 60 GPa·m.
53. The applicator according to any of the preceding items, wherein the applicator comprises a sheath enclosing the delivery tube at least partly.
54. The applicator according to item 53, wherein the sheath has a stiffness of above 0.5, 1.5 or 2 GPa·m, more preferably above 50 or 60 GPa·m.
55. The applicator according to any of the preceding items, wherein the applicator contains a volume of between 3-20 ml, preferably between 4-12 ml, such as 8 ml or 10 ml.
56. The applicator according to any of the preceding items, wherein the applicator comprises a material selected from the group of: metals, plastics, polymers, glass, glass fibers, carbon fibers, polymer fibers, composites such as fiber-reinforced materials, and combinations thereof.
57. The applicator according to any of the preceding items 2-56, wherein the pressure generating unit is a piston, a spring acting on a plate positioned at a distal end of the paste, a screw for rotating at least partly inside the delivery tube or a gas pressurization unit for providing a gas with an enhanced pressure.
58. The applicator according to any of the preceding items, wherein the delivery tube and the nozzle assembly are fluidly connected by peripheral openings.
59. The applicator according to any of the preceding items, wherein the nozzle assembly comprises at a proximal end a central wall substantially perpendicular to a longitudinal axis of the applicator or the nozzle assembly, wherein the central wall has peripheral openings connecting the delivery tube and the nozzle assembly.
60. The applicator according to any of the preceding items, wherein the nozzle assembly comprises a central wall dividing the delivery tube and the swirl unit, wherein the central wall has peripheral openings connecting the delivery tube and the nozzle assembly.
61. The applicator according to item 59 or 60, wherein the central wall has a proximally directed tip or third protrusion for directing the paste towards the peripheral openings.
62. The applicator according to any of the preceding items, wherein the swirl unit is a single unit or standalone unit.
63. The applicator according to any of the preceding items, wherein the swirl unit is integrated in the nozzle assembly.
64. The applicator according to any of the preceding items, wherein the applicator has, fully or at least partly, an outer thickness at least in one dimension or in two dimensions of less than 7 mm, preferably less than 6 mm, more preferably less than 5 mm, like e.g. 4 mm.
65. A nozzle assembly according to any of the preceding items.
66. A method for applying surgical haemostatics to a target site comprising the step of
providing an applicator according to any of the preceding items 1-65 connected to a container comprising a paste,
applying pressure on the paste in the container for feeding the paste into the applicator, and
spraying the paste through a nozzle assembly onto the target site.
67. The method according to item 66, wherein the pressure applied on the paste forms a spray pressure over the nozzle assembly that is at least 2 bar, preferably at least 5 bar, more preferably at least 10 bar, even more preferably at least 20 bar, possibly even at least 25 or 30 bar.
68. The method according to item 66 or 67, wherein the paste has a viscosity of at least 500 Pa·s or between 500 Pa·s and 8000 Pa·s, preferably between 500 Pa·s and 3500 Pa·s, such as 1500 Pa·s.

1. An applicator for delivering a paste from a container, comprising:
a delivery tube for connecting at a proximal end to the container, and
a nozzle assembly at a distal end of the delivery tube for delivering the paste as a spray, preferably as a spray of droplets or of microparticles.

2. The applicator according to claim 1, wherein the applicator comprises the container, and wherein the applicator comprises a pressure generating unit at a proximal end of the delivery tube for forcing the paste out of the container, into and through the delivery tube, to generate a spray pressure at a proximal end of the nozzle assembly.

3. The applicator according to claim 2, configured such that the spray pressure is at least 2 bar, preferably at least 5 bar, more preferably at least 10 bar, even more preferably at least 20 bar, possibly even at least 25 or 30 bar.

4. The applicator according to any of the preceding claims, wherein the paste has a viscosity of at least 500 Pa·s or between 500 Pa·s and 8000 Pa·s, preferably between 500 Pa·s and 3500 Pa·s, such as 1500 Pa·s and/or wherein the applicator is configured for delivering a spray from a paste with a viscosity of at least 500 Pa·s, or a viscosity between 500 Pa·s and 8000 Pa·s, preferably between 500 Pa·s and 3500 Pa·s, such as 1500 Pa·s.

5. The applicator according to any of the preceding claims, wherein the nozzle assembly comprises a nozzle head, preferably located in extension of the distal end of the delivery tube, the nozzle head having a spray orifice at the spray outlet.

6. The applicator according to any of the preceding claims, wherein the nozzle assembly comprises a swirl unit for creating a rotating movement of the paste.

7. The applicator according to claims 5 or 6, wherein the spray orifice comprises a paste inlet opening inwards, wherein the paste inlet is preferably converging in the distal direction e.g. cone shaped, trumpet shaped, or parabolic in cross-section, and/or wherein the spray orifice comprises a spray outlet opening outwards, wherein the spray outlet is preferably diverging in the distal direction or spraying direction e.g. cone shaped, trumpet shaped, or parabolic in cross-section.

8. The applicator according to any of the preceding claims 6-7, wherein the swirl unit comprises one or more axial guides, such as two, three or more axial guides, extending towards a paste inlet.

9. The applicator according to any of the preceding claims 6-8, wherein the swirl unit and/or the distal surface of the nozzle head comprise(s) one or more tangential guides or tangential channels, such as two, three or more tangential guides, towards a spray outlet.

10. The applicator according to claim 9, wherein the one or more tangential guides are extending non-radially.

11. The applicator according to claims 9 or 10, wherein the tangential guides are curved.

12. The applicator according to any of the preceding claims 6-10, wherein the swirl unit comprises at a distal end a pointed tip or second protrusion, preferably cone shaped, trumpet shaped or bullet shaped.

13. The applicator according to claim 11, wherein the pointed tip is at least partly located inside the spray orifice or configured to be at least partly located inside the spray orifice.

14. The applicator according to claim 12 or 13, wherein the pointed tip of the swirl unit only extends through a part of the spray orifice.

15. The applicator according to any of the preceding claims 12-14, wherein the opening angle of the pointed tip is less than 80 degrees, preferably less than 70 degrees, more preferably less than 60, most preferably around 50 degrees.

16. The applicator according to any of the preceding claims 12-15, wherein the opening angle of the pointed tip and the paste inlet have substantially the same angle.

17. The applicator according to any of the preceding claims 12-16, wherein the opening angle of the pointed tip is greater than the paste inlet.

18. The applicator according to any of the preceding claims 12-17, wherein the opening angle of the pointed tip is smaller than the paste inlet.

19. The applicator according to any of the preceding claims, wherein the applicator is adapted for insertion into a trocar.

20. The applicator according to any of the preceding claims, wherein the applicator comprises a sheath enclosing the delivery tube at least partly.

21. The applicator according to claim 20, wherein the sheath has a stiffness of above 0.5, 1.5 or 2 GPa·m, more preferably above 50 or 60 GPa·m.

22. The applicator according to claim 20 or 21, wherein the sheath is shorter than the delivery tube such that the distal end of the applicator can be manipulated by a robotic arm for directing a spray direction.

23. The applicator according to any of the preceding claims, wherein the nozzle assembly comprises at a proximal end a central wall substantially perpendicular to a longitudinal axis of the applicator or the nozzle assembly, wherein optionally the central wall has peripheral openings connecting the delivery tube and the nozzle assembly.

24. The applicator according to any of the preceding claims, wherein the nozzle assembly comprises a central wall dividing the delivery tube and the swirl unit, wherein the central wall has peripheral openings connecting the delivery tube and the nozzle assembly.

25. The applicator according to claim 23 or 24, wherein the central wall has a proximally directed tip or third protrusion for directing the paste towards the peripheral openings.

26. A nozzle assembly according to any of the preceding claims.

27. A method for applying surgical haemostatics to a target site comprising the step of
providing an applicator according to any of the preceding claims 1—connected to a container comprising a paste,
applying pressure on the paste in the container for feeding the paste into the applicator, and
spraying the paste through a nozzle assembly onto the target site.

28. The method according to claim 26, wherein the paste has a viscosity of at least 500 Pa·s or between 500 Pa·s and 8000 Pa·s, preferably between 500 Pa·s and 3500 Pa·s, such as 1500 Pa·s.
